(19) United States
(12) Patent Application Publication
(10) Pub. No.: US 2015/0218242 A1
(43) Pub. Date: Aug. 6, 2015

(54) TIF1-Gamma for Treating and Diagnosing Inflammatory Diseases

(71) Applicants: INSERM (Institut National de la Sante et de la Recherche Medicale), Paris (FR); CEA, Cedex Gif-sur-Yvette (FR); Universite Paris-Sud X1, Orsay Cedex (FR); Universite Paris Diderot-Paris 7, Paris (FR)

(72) Inventors: Paul-Henri Romeo, Fontenay aux Roses Cedex (FR); Aude Parcelier, Fontenay aux Roses Cedex (FR); Federica Ferri, Fontenay aux Roses Cedex (FR); Vanessa Petit, Fontenay aux Roses Cedex (FR); Nathalie Gault, Fontenay aux Roses Cedex (FR); Daniel Lewandowski, Fontenay aux Roses Cedex (FR)

(21) Appl. No.: 14/418,513

(22) PCT Filed: Jul. 31, 2013
(86) PCT No.: PCT/EP2013/066098

§ 371 Date: Jan. 30, 2015

(30) Foreign Application Priority Data
Jul. 31, 2012 (EP) 12305945.3

(51) Int. Cl. C07K 14/47; C12Q 1/68; A01K 67/027

(52) U.S. Cl. CPC C07K 14/4705; A01K 67/0275; C12Q 1/6883; C12Q 2600/156; C12Q 2600/136; A01K 2227/105; A01K 2267/0306

(57) ABSTRACT

The present invention relates to the prevention or treatment of inflammatory diseases. The present invention also relates to a method for screening a compound capable of promoting or restoring the resolution of inflammation and which may be useful for preventing or treating inflammatory disorders.

### FIELD OF THE INVENTION

The present invention relates to the prevention or treatment of acute and chronic inflammatory diseases. The present invention also relates to a method for screening compounds capable of promoting or restoring the resolution of inflammation and which may be useful for treating inflammatory disorders.

### BACKGROUND OF THE INVENTION

Until now, much progress has been made in elucidating the mechanisms regulating the induction and progression of inflammatory diseases leading to the development of new anti-inflammatory drugs. However, there is still a need for understanding the mechanisms governing the resolution of inflammation since they represent an important way to develop novel strategies for the therapy of inflammatory diseases.

Acute inflammation such as sepsis is a life-threaten disease that requires new anti inflammatory drugs. Chronic inflammation is a characteristic feature of many inflammatory diseases, such as rheumatoid arthritis (RA) or inflammatory bowel disease (IBD). Perturbation of the processes usually involved in resolution of inflammation is an important underlying feature of chronic inflammatory diseases and persistent inflammatory stimuli or dysregulation of mechanisms of the resolution phase results in chronic inflammation are known to be key underlying factors in the progression of a range of diseases. Thus, elucidation of the mechanisms involved in the resolution of inflammation may reveal novel therapeutic targets with important clinical applications and will be useful for providing new opportunities for resolution-based pharmacology for the treatment and prevention of inflammatory diseases. In addition, it may lead to the identification of clinically relevant biomarkers of an aberrant resolution process.

TIF1γ has been recently shown to be involved in the regulation of adult hematopoiesis through TIF1γ-mediated transcriptional repression of TALI and PU.1 target genes (Kusy et al., 2011). Based on the effects of hematopoietic tissue-targeted deletion of TIF1γ in mice, it was also shown that a decreased expression of TIF1γ in bone marrow cells induced a myelomonocytic proliferation. These results led to the identification of a decreased expression of TIF1γ in almost 40% of patients with chronic myelomonocytic leukemia (CMML) suggesting TIF1γ as a putative tumor suppressor gene candidate involved in human CMML pathogenesis and TIF1γ (TRIM33) has been identified as a new marker to diagnose CMML and as a tumor suppressor gene in this disease (as described in Aucagne et al., 2011 and US patent application No US 2011/0301110).

### SUMMARY OF THE INVENTION

In a first aspect, the present invention relates to a TIF1γ-encoding polynucleotide or a TIF1γ polypeptide for use in therapy, in particular for treating or preventing an inflammatory disease.

In a second aspect, the present invention also relates to a method for screening a compound capable of capable of promoting and/or restoring the resolution of inflammation, comprising the step consisting of identifying a compound that activates the expression of TIF1γ or identifying a compound that mimics the TIF1γ transcriptional activity.

In third aspect, the present invention further relates to a transgenic non-human animal which is TIF1γ-deficient in myeloid cells.

In another aspect, the present invention relates to a method for diagnosing or predicting an inflammatory disease, or a risk of an inflammatory disease, in a subject, said method comprising detecting a mutation in the TIF1γ gene in a sample obtained from said subject, wherein the presence of a TIF1γ mutation is indicative of an inflammatory disease or of a risk of an inflammatory disease.

In still another aspect, the present invention relates to a method for diagnosing or predicting an inflammatory disease, or a risk of an inflammatory disease, in a subject, said method comprising (i) detecting the level of expression of the TIF1γ gene in a sample obtained from said subject, and (ii) comparing said level of expression with a predetermined reference value, wherein a decrease in the level of expression of the TIF1γ gene is indicative of an inflammatory disease or of a risk of an inflammatory disease.

In still another aspect, the present invention relates to use of TIF1γ as a biomarker of inflammation in a patient.

### DETAILED DESCRIPTION OF THE INVENTION

The present invention is based on the discovery that TIF1γ regulates inflammation and more particularly the resolution of inflammation.

The inventors have shown using Mx-Cre/Tif1^{γ-/-} mice (hematopoietic specific-deletion of TIF1γ) and in Lyz-Cre/Tif1^{γ-/-} mice (myeloid specific-deletion of TIF1γ) that TIF1γ plays a major role in inflammation since these mice are highly sensible to irradiation-mediated inflammation, LPS-induced endotoxicosis and DSS-induced colitis. The inventors have also demonstrated that myeloid deletion of TIF1γ results in the failure to repress several genes such as P2rx7 and P2ry12, Pai-1, IL33 and IFNβ in macrophages (usually repressed during the resolution of inflammation).

### DEFINITIONS

Throughout the specification, several terms are employed and are defined in the following paragraphs.

As used herein, the term “TIF1γ gene” (transcriptional intermediary factor 1γ) (Gene ID: 51592), which is also known as ectodermin, TRIM33, RFG7), encodes for a nuclear protein which acts as a transcriptional regulator. TIF1γ can be from any source, but typically is a mammalian (e.g., human and non-human primate) TIF1γ, particularly a human TIF1γ. The term may also include naturally occurring TIF1γ variants and modified forms thereof. Thus, two splice variant isoforms have been described, respectively a TIF1γ alpha variant which refers to a protein of 1127 amino acids provided in the GenPept database under accession number NP_{—}056990 and is encoded by the nucleic acid sequence provided in the GenBank database under accession number NM_{—}015906 and a TIF1γ beta variant which refers to a protein of 1110 amino acids provided in the GenPept database under accession number NP_{—}148980 and which is encoded by the nucleic acid sequence provided in the GenBank database under accession number NM_{—}033020.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e., the nucleotide sequence encodes an amino acid sequence for that polypeptide, protein or enzyme. A coding sequence for a protein may include a start codon (usually ATG) and a stop codon.

As used herein, the term “gene” refers to a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed. Some genes, which are not structural genes, may be transcribed from DNA to RNA, but are not translated into an amino acid sequence. Other genes may function as regulators of structural genes or as regulators of DNA transcription. In particular, the term gene may be intended for the genomic sequence encoding a protein, i.e. a sequence comprising regulator, promoter, intron and exon sequences.

As used herein, the terms “mutant” and “mutation” mean any detectable change in genetic material, e.g. DNA, RNA, cDNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. Generally a mutation is identified in a subject by comparing the sequence of a nucleic acid or polypeptide expressed by said subject with the corresponding nucleic acid or polypeptide expressed in a control population. A mutation in the genetic material may also be “silent”, i.e. the mutation does not result in an alteration of the amino acid sequence of the expression product.

The term “biomarker”, as used herein, refers generally to a molecule, i.e., a gene (or nucleic acid encoding said gene), protein, the expression of which in a biological sample from a patient can be detected by standard methods in the art (as well as those disclosed herein), and is predictive or denotes a condition of the patient from which it was obtained.

As used herein, the term “inflammatory diseases” refers to any disease and disorder having etiologies associated with a systemic or local abnormal and/or uncontrolled inflammatory response. For instance, over-expression of proinflammatory cytokines without proper controls leads to a variety of inflammatory diseases and disorders. This term includes both acute inflammatory diseases and chronic inflammatory diseases. Examples of inflammatory disease include, but are not limited to, rheumatoid arthritis (RA), inflammatory bowel disease (IBD) including ulcerative colitis (UC) and Crohn's Disease (CD), diabetes, sepsis, allergic inflammation, asthma, chronic obstructive pulmonary disease (COPD), atherosclerosis, multiple sclerosis and systemic lupus erythematosus (SLE).

### Diagnostic Methods of the Invention

The inventors have shown that myeloid specific deletion of the TIF1γ gene is associated with a defective process of resolution of the inflammation in a new mouse model (more precisely is associated with a defective repression of several genes involved in inflammation such as P2rx7 and P2ry12, Pai-1, IL-33 and IFNβ leading to a deleterious over-expression of these genes). Defective TIF1γ is thus thought to be involved in the pathological process of inflammation and conversely TIF1γ promotes the resolution of the inflammation in a physiological state.

Therefore, the present invention relates to a method for diagnosing or predicting an inflammatory disease, or a risk of an inflammatory disease, in a subject, said method comprising detecting a mutation in the TIF1γ gene in a sample obtained from said subject, wherein the presence of a TIF1γ mutation is indicative of an inflammatory disease or of a risk of an inflammatory disease.

A TIF1γ mutation according to the invention may be found in a regulating region of TIF1γ gene (e.g. a promoter sequence, a binding site for transcription factor) or in exons that encode the TIF1γ protein. Typically the TIF1γ mutation is a mutation which results in a decreased expression of TIF1γ or the TIF1γ mutation is a nonsense mutation which results in a truncated non functional TIF1γ protein or the TIF1γ mutation is a mutation which results in a point mutated non functional TIF1γ protein.

In an embodiment, the TIF1γ mutation is heterozygous.

In another embodiment, the TIF1γ mutation is homozygous. As used herein, the term, “homozygous TIF1γ mutation” refers to a subject whose two alleles of the TIF1γ gene are mutated. The expression thus encompasses both homozygous mutations strico sense (wherein the same mutation is present on both alleles) and composite heterozygous mutation (wherein each allele presents a different mutation).

Such a mutation may be detected by analyzing TIF1γ nucleic acid molecule. In the context of the invention, TIF1γ nucleic acid molecules include mRNA, genomic DNA, cDNA derived from mRNA. DNA or RNA can be single stranded or double stranded. These may be utilized for detection by amplification and/or hybridization with a probe, for instance.

The nucleic acid sample may be obtained from any cell source or tissue biopsy. Non-limiting examples of cell sources available include without limitation blood cells, buccal cells, epithelial cells, fibroblasts, or any cells present in a tissue obtained by biopsy. Cells may also be obtained from body fluids, such as blood, plasma, serum, lymph, etc. DNA may be extracted using any methods known in the art, such as described in Sambrook et al., 1989. RNA may also be isolated, for instance from tissue biopsy, using standard methods well known to the one skilled in the art such as guanidium thiocyanate-phenol-chloroform extraction.

TIF1γ mutations may be detected in a RNA or DNA sample, preferably after amplification. For instance, the isolated RNA may be subjected to coupled reverse transcription and amplification, such as reverse transcription and amplification by polymerase chain reaction (RT-PCR), using specific oligonucleotide primers that are specific for a mutated site or that enable amplification of a region containing the mutated site. According to a first alternative, conditions for primer annealing may be chosen to ensure specific reverse transcription (where appropriate) and amplification; so that the appearance of an amplification product be a diagnostic of the presence of a particular TIF1γ mutation. Otherwise, RNA may be reverse-transcribed and amplified, or DNA may be amplified, after which a mutated site may be detected in the amplified sequence by hybridization with a suitable probe or by direct sequencing, or any other appropriate method known in the art. For instance, a cDNA obtained from RNA may be cloned and sequenced to identify a mutation in TIF1γ sequence.

Actually numerous strategies for genotype analysis are available (Antonarakis et al., 1989; Cooper et al., 1991; Grompe, 1993). Briefly, the nucleic acid molecule may be tested for the presence or absence of a restriction site. When a base substitution mutation creates or abolishes the recognition site of a restriction enzyme, this allows a simple direct PCR test for the mutation. Further strategies include, but are not limited to, direct sequencing, restriction fragment length polymorphism (RFLP) analysis; hybridization with allele-specific oligonucleotides (ASO) that are short synthetic probes which hybridize only to a perfectly matched sequence under suitably stringent hybridization conditions; allele-specific PCR; PCR using mutagenic primers; ligase-PCR, HOT cleavage; denaturing gradient gel electrophoresis (DGGE), temperature denaturing gradient gel electrophoresis (TGGE), single-stranded conformational polymorphism (SSCP) and denaturing high performance liquid chromatography (Kuklin et al., 1997). Direct sequencing may be accomplished by any method, including without limitation chemical sequencing, using the Maxam-Gilbert method; by enzymatic sequencing, using the Sanger method; mass spectrometry sequencing; sequencing using a chip-based technology; and real-time quantitative PCR. Preferably, DNA from a subject is first subjected to amplification by polymerase chain reaction (PCR) using specific amplification primers. However several other methods are available, allowing DNA to be studied independently of PCR, such as the rolling circle amplification (RCA), the InvaderTMassay, or oligonucleotide ligation assay (OLA). OLA may be used for revealing base substitution mutations. According to this method, two oligonucleotides are constructed that hybridize to adjacent sequences in the target nucleic acid, with the join sited at the position of the mutation. DNA ligase will covalently join the two oligonucleotides only if they are perfectly hybridized.

Therefore, useful nucleic acid molecules, in particular oligonucleotide probes or primers, according to the present invention include those which specifically hybridize the regions where the mutations are located.

Oligonucleotide probes or primers may contain at least 10, 15, 20 or 30 nucleotides. Their length may be shorter than 400, 300, 200 or 100 nucleotides.

In a further embodiment, a subset of mutations of the TIF1γ gene may be detected at the protein level.

Such a mutation may be detected according to any appropriate method known in the art. In particular a sample, such as a tissue biopsy, obtained from a subject may be contacted with antibodies specific of a mutated form of TIF1γ, i.e. antibodies that are capable of distinguishing between a mutated form of TIF1γ and the wild-type protein (or any other protein), to determine the presence or absence of a TIF1γ specified by the antibody and also to detect abnormal cellular localisation of TIF1γ (e.g. cytoplasmic localisation) caused by particular mutations such as mutations in the bromodomain.

Antibodies that specifically recognize a mutated TIF1γ also make part of the invention. The antibodies are specific of mutated TIF1γ, which is to say they do not cross-react with the wild-type TIF1γ.

The antibodies of the present invention may be monoclonal or polyclonal antibodies, single chain or double chain, chimeric antibodies, humanized antibodies, or portions of an immunoglobulin molecule, including those portions known in the art as antigen binding fragments Fab, Fab′, F(ab′)2 and F(v). They can also be immunoconjugated, e.g. with a toxin, or labelled antibodies.

Whereas polyclonal antibodies may be used, monoclonal antibodies are preferred for they are more reproducible in the long run.

Procedures for raising “polyclonal antibodies” are also well known. Polyclonal antibodies can be obtained from serum of an animal immunized against the appropriate antigen, which may be produced by genetic engineering for example according to standard methods well-known by one skilled in the art. Typically, such antibodies can be raised by administering mutated TIF1γ subcutaneously to New Zealand white rabbits which have first been bled to obtain pre-immune serum. The antigens can be injected at a total volume of 100 μl per site at six different sites. Each injected material may contain adjuvants with or without pulverized acrylamide gel containing the protein or polypeptide after SDS-polyacrylamide gel electrophoresis. The rabbits are then bled two weeks after the first injection and periodically boosted with the same antigen three times every six weeks. A sample of serum is then collected 10 days after each boost. Polyclonal antibodies are then recovered from the serum by affinity chromatography using the corresponding antigen to capture the antibody. This and other procedures for raising polyclonal antibodies are disclosed by Harlow et al. (1988).

A “monoclonal antibody” in its various grammatical forms refers to a population of antibody molecules that contains only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g. a bispecific monoclonal antibody. Although historically a monoclonal antibody was produced by immortalization of a clonally pure immunoglobulin secreting cell line, a monoclonally pure population of antibody molecules can also be prepared by the methods of the present invention.

Laboratory methods for preparing monoclonal antibodies are well known in the art (see, for example, Harlow et al., 1988). Monoclonal antibodies (mAbs) may be prepared by immunizing purified mutated TIF1γ into a mammal, e.g. a mouse, rat, human and the like mammals. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in Kohler and Milstein (1975).

While mAbs can be produced by hybridoma culture the invention is not to be so limited. Also contemplated is the use of mAbs produced by an expressing nucleic acid cloned from a hybridoma of this invention. That is, the nucleic acid expressing the molecules secreted by a hybridoma of this invention can be transferred into another cell line to produce a transformant. The transformant is genotypically distinct from the original hybridoma but is also capable of producing antibody molecules of this invention, including immunologically active fragments of whole antibody molecules, corresponding to those secreted by the hybridoma. See, for example, U.S. Pat. No. 4,642,334 to Reading; European Patent Publications No. 0239400 to Winter et al. and No. 0125023 to Cabilly et al.

Antibody generation techniques not involving immunisation are also contemplated such as for example using phage display technology to examine naive libraries (from non-immunised animals); see Barbas et al. (1992), and Waterhouse et al. (1993).

Antibodies raised against mutated TIF1γ may be cross reactive with wild-type TIF1γ. Accordingly a selection of antibodies specific for mutated TIF1γ is required. This may be achieved by depleting the pool of antibodies from those that are reactive with the wild-type TIF1γ, for instance by submitting the raised antibodies to an affinity chromatography against wild-type TIF1γ.

Alternatively, binding agents other than antibodies may be used for the purpose of the invention. These may be for instance aptamers, which are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by EXponential enrichment (SELEX) of a random sequence library, as described in Tuerk C. and Gold L., 1990. The random sequence library is obtainable by combinatorial chemical synthesis of DNA. In this library, each member is a linear oligomer, eventually chemically modified, of a unique sequence. Possible modifications, uses and advantages of this class of molecules have been reviewed in Jayasena S. D., 1999. Peptide aptamers consists of a conformationally constrained antibody variable region displayed by a platform protein, such as E. coli Thioredoxin A that are selected from combinatorial libraries by two hybrid methods (Colas et al., 1996).

Probe, primers, aptamers or antibodies of the invention may be labelled with a detectable molecule or substance, such as a fluorescent molecule, a radioactive molecule or any others labels known in the art. Labels are known in the art that generally provide (either directly or indirectly) a signal.

The term “labelled”, with regard to the probe, primers, aptamers or antibodies of the invention, is intended to encompass direct labelling of the probe, primers, aptamers or antibodies of the invention by coupling (i.e., physically linking) a detectable substance to the the probe, primers, aptamers or antibodies of the invention, as well as indirect labeling of the probe, primers, aptamers or antibodies of the invention by reactivity with another reagent that is directly labeled. Other examples of detectable substances include but are not limited to radioactive agents or a fluorophore (e.g. fluorescein isothiocyanate (FITC) or phycoerythrin (PE) or Indocyanine (Cy5)). Examples of indirect labeling include detection of a primary antibody using a fluorescently labeled secondary antibody and end-labeling of a DNA probe with biotin such that it can be detected with fluorescently labeled streptavidin. An antibody or aptamer of the invention may be labelled with a radioactive molecule by any method known in the art. For example radioactive molecules include but are not limited radioactive atom for scintigraphic studies such as 1123, 1124, In111, Re186, Re188.

Alternatively, the present invention relates to a method for diagnosing or predicting an inflammatory disease, or a risk of an inflammatory disease, in a subject, said method comprising detecting an epigenetic modification of the TIF1γ gene in a sample obtained from said subject, wherein the presence of an epigenetic modification of the TIF1γ gene is indicative of an inflammatory disease or of a risk of an inflammatory disease.

In one embodiment, the epigenetic modification of the TIF1γ gene is DNA methylation.

In a particular embodiment, the epigenetic modification of the TIF1γ gene is the DNA methylation of the TIF1γ gene promoter.

One of the principle mechanisms of epigenetic gene regulation is DNA methylation in which methyl groups are added to CpG sites within DNA sequences. Levels of DNA methylation are correlated with gene expression and low levels of methylation are typically associated with increased gene expression and high levels with reduced expression or gene silencing. Thus, an epigenetic modification of the TIF1γ gene such as DNA methylation of the TIF1γ gene promoter results in a decreased expression of TIF1γ. DNA methylation of the TIF1γ gene promoter can be monitored using bisulfite DNA treatment and sequencing. Briefly, genomic DNA isolated from cells of interest is modified by bisulfite treatment according to the manufacturer's instructions (MethylDetector, Active Motif). Converted TIFγ promoter DNA is identified by PCR with specific primers and direct sequencing reaction is performed using standard conditions according to the manufacturer's instructions (Applied Biosystems).

The present invention also relates to a method for diagnosing or predicting an inflammatory disease, or a risk of an inflammatory disease, in a subject, said method comprising (i) detecting expression level of the TIF1γ gene in a sample obtained from said subject, and (ii) comparing said level of expression with a predetermined reference value, wherein a decrease in expression level of the TIF1γ gene is indicative of an inflammatory disease or of a risk of an inflammatory disease.

As used herein, the term “sample” has its general meaning in the art and refers to any sample which may be obtained from a patient. Examples of such samples include fluids, tissues, cell samples, organs, biopsies, etc. A preferred sample is a sample containing monocytes and/or macrophages such as a blood sample (whole blood, serum or plasma) or biopsies that can be used for immunohistochemistry.

At step ii) of the method, the value for the expression level of TIF1γ gene which is obtained at the end of step a) is compared with a predetermined reference value for the expression level of TIF1γ.

As used herein, the term “predetermined reference value” refers to the amount of TIF1γ in biological samples obtained from the general population or from a selected population of subjects. The predetermined reference value can be a threshold value or a range. For example, the selected population may be comprised of apparently healthy subjects, such as individuals who have not previously had any sign or symptoms indicating the presence of an inflammatory disease.

Methods for Determining the Expression Level of the Biomarker of the Invention:

Determination of the expression level of TIF1γ gene may be performed by a variety of techniques. Generally, the expression level as determined is a relative expression level. For example, the determination comprises contacting the biological sample with selective reagents such as probes, primers or ligands, and thereby detecting the presence, or measuring the amount, of polypeptide or nucleic acids of interest originally in said biological sample. Contacting may be performed in any suitable device, such as a plate, microtiter dish, test tube, well, glass, column, and so forth. In specific embodiments, the contacting is performed on a substrate coated with the reagent, such as a nucleic acid array or a specific ligand array. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as a nucleic acid hybrid or an antibody-antigen complex, to be formed between the reagent and the nucleic acids or polypeptides of the biological sample.

As used herein, the term “determining” includes qualitative and/or quantitative detection (i.e. detecting and/or measuring the expression level) with or without reference to a control or a predetermined value. As used herein, “detecting” means determining if TIF1γ is present or not in a biological sample and “measuring” means determining the amount of TIF1γ in a biological sample. Typically the expression level may be determined for example by RT-PCR or immunohistochemistry (IHC) performed on a biological sample.

In a particular embodiment, the expression level may be determined by determining the quantity of mRNA.

Methods for determining the quantity of mRNA are well known in the art. For example the nucleic acid contained in the biological samples (e.g., cell or tissue prepared from the patient) is first extracted according to standard methods, for example using lytic enzymes or chemical solutions or extracted by nucleic-acid-binding resins following the manufacturer's instructions. The extracted mRNA is then detected by hybridization (e.g., Northern blot analysis) and/or amplification (e.g., RT-PCR). Quantitative or semi-quantitative RT-PCR is preferred. Real-time quantitative or semi-quantitative RT-PCR is particularly advantageous.

Other methods of Amplification include ligase chain reaction (LCR), transcription-mediated amplification (TMA), strand displacement amplification (SDA) and nucleic acid sequence based amplification (NASBA).

Nucleic acids having at least 10 nucleotides and exhibiting sequence complementarity or homology to the mRNA of interest herein find utility as hybridization probes or amplification primers. It is understood that such nucleic acids need not be identical, but are typically at least about 80% identical to the homologous region of comparable size, more preferably 85% identical and even more preferably 90-95% identical.

In certain embodiments, it will be advantageous to use nucleic acids in combination with appropriate means, such as a detectable label, for detecting hybridization. A wide variety of appropriate indicators are known in the art including, fluorescent, radioactive, enzymatic or other ligands (e. g. avidin/biotin).

Probes typically comprise single-stranded nucleic acids of between 10 to 1000 nucleotides in length, for instance of between 10 and 800, more preferably of between 15 and 700, typically of between 20 and 500. Primers typically are shorter single-stranded nucleic acids, of between 10 to 25 nucleotides in length, designed to perfectly or almost perfectly match a nucleic acid of interest, to be amplified. The probes and primers are “specific” to the nucleic acids they hybridize to, i.e. they preferably hybridize under high stringency hybridization conditions (corresponding to the highest melting temperature Tm, e.g., 50% formamide, 5× or 6×SCC. SCC is a 0.15 M NaCl, 0.015 M Na-citrate).

The nucleic acid primers or probes used in the above detection, staging, screening and monitoring methods may be assembled as a kit. Such a kit includes consensus primers and molecular probes. A particular kit also includes the components necessary to determine if amplification has occurred. The kit may also include, for example, PCR buffers and enzymes; positive control sequences, reaction control primers; and instructions for amplifying and detecting the specific sequences.

Accordingly, the present invention also relates to a kit for performing a method above-mentioned, wherein said kit comprises means for determining the expression level of the TIF1γ gene in a biological sample obtained from said subject.

In a particular embodiment, the methods of the invention comprise the steps of providing total RNAs extracted from a biological sample such a blood sample and subjecting the RNAs to amplification and hybridization to specific probes, more particularly by means of a quantitative or semi-quantitative RT-PCR.

In another particular embodiment, the expression level is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the expression level, a biological sample from a test subject, optionally first subjected to a reverse transcription, is labelled and contacted with the microarray in hybridization conditions, leading to the formation of complexes between target nucleic acids that are complementary to probe sequences attached to the microarray surface. The labelled hybridized complexes are then detected and can be quantified or semi-quantified. Labelling may be achieved by various methods, e.g. by using radioactive or fluorescent labelling. Many variants of the microarray hybridization technology are available to the man skilled in the art (see e.g., Hoheisel, Nature Reviews, Genetics, 2006, 7:200-210).

In this context, the invention further provides a DNA chip comprising a solid support which carries nucleic acids that are specific to the TIF1γ gene.

Other methods for determining the expression level of said genes include the determination of the quantity of proteins encoded by TIF1γ gene.

Such methods comprise contacting the sample with a binding partner capable of selectively interacting with a marker protein present in the sample. The binding partner is generally an antibody that may be polyclonal or monoclonal, preferably monoclonal. Monoclonal antibodies directed against TIF1γ are well known from the skilled man in the art such as the antibodies commercialized by ABD SEROTEC, ABGENT, ABNOVA CORPORATION, LIFESPAN BIOSCIENCES, SANTA CRUZ BIOTECHNOLOGY Inc., or SIGMA-ALDRICH.

It should be further noted that the binding partner may also be an aptamer.

The presence of the protein of interest may be detected using standard electrophoretic and immunodiagnostic techniques, including immunoassays such as competition, direct reaction, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith. Labels are known in the art that generally provide (either directly or indirectly) a signal. As used herein, the term “labelled” with regard to the antibody or aptamer, is intended to encompass direct labelling of the antibody or aptamer by coupling (i.e., physically linking) a detectable substance, such as a radioactive agent or a fluorophorc (e.g. fluorescein isothiocyanate (FITC) or phycoerythrin (PE) or indocyanine (Cy5), to the antibody or aptamer, as well as indirect labelling of the probe or antibody (e.g., horseradish peroxidise, HRP) by reactivity with a detectable substance. An antibody or aptamer may be also labelled with a radioactive molecule by any method known in the art. For example, radioactive molecules include but are not limited radioactive atom for scintigraphic studies such as I123, I124, In111, Re186 and Re188. The aforementioned assays generally involve separation of unbound protein in a liquid phase from a solid phase support to which antigen-antibody complexes are bound. Solid supports which may be used in the practice of the invention include substrates such as nitrocellulose (e.g., in membrane or microtiter well form); polyvinylchloride (e.g., sheets or microtiter wells); polystyrene latex (e.g., beads or microtiter plates); polyvinylidine fluoride; diazotized paper; nylon membranes; activated beads, magnetically responsive beads, etc.

More particularly, an ELISA method may be used, wherein the wells of a microtiter plate are coated with an antibody against the protein to be tested. A biological sample containing or suspected of containing the marker protein is then added to the coated wells. After a period of incubation sufficient to allow the formation of antibody-antigen complexes, the plate (s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

Alternatively, an immunohistochemistry (IHC) method may be used.

IHC specifically provides a method of detecting a target protein in a biological sample or tissue specimen in situ. The overall cellular integrity of the sample is maintained in IHC, thus allowing detection of both the presence and location of the target of interest. Typically a biological sample is fixed with formalin, embedded in paraffin and cut into sections for staining and subsequent inspection by light microscopy. Current methods of IHC use either direct labelling or secondary antibody-based or hapten-based labelling. Examples of known IHC systems include, for example, EnVision™ (DakoCytomation), Powervision® (Immunovision, Springdale, Ariz.), the NBA™ kit (Zymed Laboratories Inc., South San Francisco, Calif.), HistoFine® (Nichirei Corp, Tokyo, Japan).

It should be further noted that when a decrease in the level of expression of the TIF1γ gene (mRNA or protein) is detected, it may be useful to determine the presence of an epigenetic modification of the TIF1γ gene (e.g. the DNA methylation of the TIF1γ gene promoter) and thus determine the reason of this decrease. Thus, in one embodiment, the method for diagnosing or predicting an inflammatory disease, or a risk of an inflammatory disease according to the invention comprises the following steps of (i) detecting a decrease in expression level of the TIF1γ gene in a sample obtained from said subject as described above, and (ii) detecting an epigenetic modification of the TIF1γ gene in said sample.

Moreover, as previously mentioned, some mutations may lead to an abnormal cellular localisation of TIF1γ. Thus, the present invention further relates to a method for diagnosing or predicting an inflammatory disease, or a risk of an inflammatory disease, in a subject, said method comprising determining the cellular localization of TIF1γ (preferably subcellular localization), in a sample obtained from said subject, wherein an abnormal localization of the TIF1γ is indicative of an inflammatory disease or of a risk of an inflammatory disease.

In one embodiment, the abnormal localization of the TIF1γ is a cytoplasmic localization.

Methods for determining the cellular localization of TIF1γ include immunohistochemistry (IHC) as above-described.

Examples of samples include samples containing cells such as fluids, tissues, cell samples, organs, biopsies, etc. A preferred sample is a sample containing monocytes and/or macrophages such as a blood sample (whole blood or plasma).

Another aspect of the present invention provides a method for determining the inflammatory status of a patient affected with an inflammatory disease, particularly with a chronic inflammatory disease or a method for distinguishing between a stabilized non inflammatory form and a inflammatory form (disease flares) of a chronic inflammatory disease in a patient previously diagnosed with said chronic inflammatory disease, said method comprising (i) detecting expression level of the TIF1γ gene in a sample obtained from said subject, and (ii) comparing said level of expression with a predetermined reference value, wherein a decrease in expression level of the TIF1γ gene is indicative of an inflammatory form of a chronic inflammatory disease.

A further aspect of the invention relates to the use of TIF1γ as a biomarker of inflammation, in particular acute inflammation, in a patient.

In one embodiment, the patient has been previously diagnosed with an inflammatory disease (e.g. chronic inflammatory diseases such as systemic lupus erythematosus).

Accordingly, TIF1γ may be used in diagnosis, prognosis, evaluating the inflammatory status or monitoring the effects of therapy in diseases characterized by inflammation.

### Therapeutic Methods of the Invention

In a further object, the invention relates to use, methods and pharmaceutical compositions for use in the prevention or treatment of an inflammatory disease.

Gene therapy is a convenient way to prevent or treat an inflammatory disease as it enables the provision of a constant supply of polypeptide or correction of the defective gene, for example as discussed below.

Gene therapy may be carried out by means of supplementation of cells lacking a functional TIF1γ polypeptide with a wild type TIF1γ gene product. Production of a suitable gene product may be achieved using recombinant techniques. For example, a suitable vector may be inserted into a host cell and expressed in that cell.

The present invention further relates to a method for preventing or treating an inflammatory disease which comprises the step of administering a subject in need thereof with a TIF1γ polynucleotide, i.e. a nucleic acid sequence that encodes a wild-type TIF1γ, so that TIF1γ is expressed in vivo by the cells of the subject that have been transfected with said polynucleotide. Accordingly, said method leads to an overexpression of wild-type TIF1γ which compensates expression of defective mutated TIF1γ.

The present invention also relates to a TIF1γ-encoding polynucleotide or a TIF1γ polypeptide for use in therapy and more particularly a TIF1γ-encoding polynucleotide or a TIF1γ polypeptide for use in the prevention or treatment of an inflammatory disease.

In one embodiment, the inflammatory disease is induced by at least one protein encoded by up-regulated genes in TIF1γ^{-/-} macrophages selected from the group including CD3001f, Gsto1, P2rx7, P2ry12, P2ry13, Ptgis, TNFSF4, IL33, IFNβ, ANGPTL3, Cadm1, CamK2a, Esr1, PAI-1, FLRT3 and SPP1.

In another embodiment, the inflammatory disease is induced by at least one protein encoded by down-regulated genes in TIF1γ^{-/-} macrophages selected from the group including EGR1, KLF3, SMAD6, CX3CR1, CXCL5, ENPP2, 1TGA9, OSM and PLA2G7.

Said TIF1γ polynucleotide is administered in a therapeutically effective amount.

Preferably the TIF1γ polynucleotide sequence according to the invention is associated with elements that enable for regulation of its expression, such as a promoter sequence.

Such a nucleic acid may be in the form of a vector. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses (AAV)), which serve equivalent functions.

The TIF1γ polynucleotide may be introduced into a target cell by means of any procedure known for the delivery of nucleic acids to the nucleus of cells, ex vivo, on cells in culture or removed from an animal or a patient, or in vivo.

Ex vivo introduction may be performed by any standard method well known by one skilled in the art, e.g. transfection, electroporation, microinjection, transduction, cell fusion, DEAE dextran, calcium phosphate precipitation, or use of a gene gun.

The TIF1γ polynucleotide can also be introduced ex vivo or in vivo by lipofection. In certain embodiments, the use of liposomes and/or nanoparticles is contemplated for the introduction of the donor nucleic acid targeting system into host cells.

Nanocapsules can generally entrap compounds in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core.

Synthetic cationic lipids designed to limit the difficulties and dangers encountered with liposome mediated transfection can be used to prepare liposomes for in vivo transfection of a gene encoding a marker. The use of cationic lipids may promote encapsulation of negatively charged nucleic acids, and also promote fusion with negatively charged cell membranes (Feigner et al., 1989).

Alternatively, one of the simplest and the safest way to deliver TIF1γ polynucleotide across cell membranes in vivo may involve the direct application of high concentration free or naked polynucleotides (typically mRNA or DNA). By “naked DNA (or RNA)” is meant a DNA (RNA) molecule which has not been previously complexed with other chemical moieties. Naked DNA uptake by animal cells may be increased by administering the cells simultaneously with excipients and the nucleic acid. Such excipients are reagents that enhance or increase penetration of the DNA across cellular membranes and thus delivery to the cells delivery of the therapeutic agent. Various excipients have been described in the art, such as surfactants, e.g. a surfactant selected form the group consisting of Triton X-100, sodium dodecyl sulfate, Tween 20, and Tween 80; bacterial toxins, for instance streptolysin 0, cholera toxin, and recombinant modified labile toxin of E coli; and polysaccharides, such as glucose, sucrose, fructose, or maltose, for instance, which act by disrupting the osmotic pressure in the vicinity of the cell membrane. Other methods have been described to enhance delivery of free polynucleotides, such as blocking of polynucleotide inactivation via endo- or exonucleolytic cleavage by both extra- and intracellular nucleases.

Alternatively, the present invention also provides a method for treating or preventing inflammatory disease which comprises the step of administering a subject in need thereof with a wild-type TIF1γ polypeptide.

Knowing the amino acid sequence of the desired sequence, one skilled in the art can readily produce said polypeptides, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions.

Alternatively, the polypeptides of the invention can be synthesized by recombinant DNA techniques as is now well-known in the art. For example, these fragments can be obtained as DNA expression products after incorporation of DNA sequences encoding the desired (poly)peptide into expression vectors and introduction of such vectors into suitable eukaryotic or prokaryotic hosts that will express the desired polypeptide, from which they can be later isolated using well-known techniques.

Polypeptides of the invention can be use in an isolated (e.g., purified) form or contained in a vector, such as a membrane or lipid vesicle (e.g. a liposome).

Alternatively, the polypeptides may be fused with a cell penetrating peptide in order to obtain an increased capacity to cross membrane barriers. The terms “cell penetrating peptide” or “CPP” are used interchangeably and refer to cationic cell penetrating peptides, also called transport peptides, carrier peptides, or peptide transduction domains. The CPP have the capability of inducing cell penetration of a polypeptide fused to the CPP within 30%, 40%, 50%, 60%, 70%, 80%, 90%, or 100% of cells of a given cell culture population, including all integers in between, and allow macromolecular translocation within multiple tissues in vivo upon systemic administration. A cell-penetrating peptide may also refers to a peptide which, when brought into contact with a cell under appropriate conditions, passes from the external environment in the intracellular environment, including the cytoplasm, organelles such as mitochondria, or the nucleus of the cell, in conditions significantly greater than passive diffusion. Such penetrating peptides may be those described in Fonseca S. B. et al., Advanced Drug Delivery Reviews, 2009, 61: 953-964. Non-limiting examples of CPP available include Tat peptide, polyarginines peptide, HA2-R9 peptide, Penetratin peptide, Transportan peptide, Vectocell® peptide, maurocalcine peptide, decalysine peptide and Antp peptide. As used herein, the term “fusion” refers to the combination of amino acid sequences of different origin in one polypeptide chain by in-frame combination of their coding nucleotide sequences.

### Screening Methods

In another aspect, the present invention relates to a method for screening a compound capable of inducing, promoting and/or restoring the resolution of inflammation, comprising the step consisting of identifying a compound that activates TIF1γ expression or identifying a compound that mimics the TIF1γ transcriptional activity.

Methods for Identifying a Compound that Activates the Expression of TIF1γ:

A particular aspect of the invention relates to a method for identifying a compound that enhances the expression of TIF1γ comprising determining whether the test compound enhances the expression of TIF1γ.

In one embodiment, the method comprises the steps of i) contacting the test compound with a cell transfected with a reporter gene operatively linked to all or part of the regulatory sequences (promoter, enhancer or silencer) of the gene encoding for TIF1γ, ii) assessing the level of expression of said reporter gene, and iii) identifying the test compound which activates the expression of said reporter gene.

Abroad variety of host-expression vector systems may be utilized to express the genes used in the method. These include, but are not limited to, mammalian cell systems such as human cell lines derived from myeloid cells including monocytes and macrophages (e.g. monocytic cell lines THP-1 and AML-193). Additional host-expression vector systems include, but are not limited to, microorganisms such as bacteria (e.g., E. coli or B. subtilis) transformed with recombinant bacteriophage DNA, plasmid DNA, or cosmid DNA expression vectors containing PTK or adaptor protein coding sequences; yeast (e.g., Saccharomyces, Pichia) transformed with recombinant yeast expression vectors containing the protein or peptide coding sequences; insect cell systems, such as Sf9 or Sf21 infected with recombinant virus expression vectors (e.g., baculovirus) containing the coding sequences of the reporter gene operatively linked to all or part of the promoter and/or enhancer and/or silencer of the gene encoding for TIF1γ; amphibian cells, such as Xenopus oocytes; or plant cell systems infected with recombinant virus expresssion vectors (e.g., cauliflower mosaic virus, CaMV; tobacco mosaic virus, TMV) or transformed with recombinant plamid expression vectors (e.g., Ti plasmid) containing the coding sequences of the reporter gene operatively linked to all or part of the promoter and/or enhancer and/or silencer of the gene encoding for TIF1γ. Culture conditions for each of these cell types is specific and is known to those familiar with the art.

DNA encoding proteins to be assayed can be transiently or stably expressed in the cell lines by several methods known in the art, such as, calcium phosphate-mediated, DEAE-dextran mediated, liposomal-mediated, viral-mediated, electroporation-mediated and microinjection delivery. Each of these methods may require optimization of assorted experimental parameters depending on the DNA, cell line, and the type of assay to be subsequently employed.

In addition native cell lines that naturally carry and express the nucleic acid sequences for the TIF1γ protein may be used.

In a particular embodiment, the invention is directed to a method, which comprises the steps of i) contacting the test compound with a cell capable of expressing the gene encoding for TIF1γ, ii) assessing the level of expression of said gene, and iii) identifying the test compound which activates the expression of said gene. In one embodiment, the level of expression is assessed by determining the level of transcription of said gene. In a further embodiment, the determination of the level of translation of said gene is effected by means of an immunoassay.

Determination of the expression level of a gene can be performed by a variety of techniques as above mentioned.

Alternatively, in another embodiment, the invention is directed to a method for screening a compound capable of capable of promoting and/or restoring the resolution of inflammation, comprising the step consisting of identifying a compound that suppresses TIF1γ silencing. As previously indicated, an epigenetic modification of the TIF1γ gene such as the DNA methylation of the TIF1γ gene promoter may result in a reduced expression of TIF1γ or gene silencing. Compound that alleviates DNA methylation of the TIFIy gene promoter will be identify using cell lines.

Methods for Identifying a Compound that Mimics the TIF1γ Transcriptional Activity:

Another particular aspect of the invention relates to a method for identifying a compound that mimics the TIF1γ transcriptional activity comprising determining whether the test compound inhibits the expression of at least one TIF1γ-target gene selected from the group consisting of TIF1γ target genes including CD3001f, Gsto1, P2rx7, P2ry12, P2ry13, Ptgis, TNFSF4, IFNβ, IL33, ANGPTL3, Cadm1, CamK2a, Esr1, PAI-1, FLRT3 and SPP1.

In one embodiment, the method comprises the steps of i) contacting the test compound with a cell transfected with a reporter gene operatively linked to all or part of the promoter and/or enhancer and/or silencer of the gene encoding for a TIF1γ-target gene (wild type) or with the same reporter gene but mutated on the TIF1γ binding sites (mutated), ii) assessing the level of expression of said reporter genes, and iii) identifying the test compound which inhibits the expression of said wild type but not mutated reporter gene.

In one particular embodiment, the TIF1γ-target gene is IFNβ.

Alternatively, another particular aspect of the invention relates to an method for identifying a compound that mimics the TIF1γ transcriptional activity comprising determining whether the test compound enhances the expression of at least one TIF1γ-target gene selected from the group consisting of EGR1, KLF3, SMAD6, CX3CR1, CXCL5, ENPP2, ITGA9, OSM and PLA2G7.

In one embodiment, the method comprises the steps of i) contacting the test compound with a cell transfected with a reporter gene operatively linked to all or part of the promoter and/or enhancer and/or silencer of the gene encoding for a TIF1γ-target gene (wild type) or with the same reporter gene but mutated on the TIF1γ binding sites (mutated), ii) assessing the level of expression of said reporter genes, and iii) identifying the test compound which inhibits the expression of said wild type but not mutated reporter gene.

All the TIF1γ target genes above-listed and those defined in the Table 1 (in Example 3 below) are known per se by the skilled man in the art.

Determination of the expression level of a gene can be performed by a variety of techniques as above mentioned.

Preferably, the test compound used in the method for identifying a compound that mimics the TIF1γ transcriptional activity according to the invention has previously been identified as a compound that activates TIF1γ expression as described above.

A particular aspect of the present invention also relates to method for screening a compound useful for mimicking the TIF1γ transcriptional repression activity comprising the steps consisting of (a) selecting a test compound by performing at least one method as described above (b) determining whether said compound mimics the TIF1γ transcriptional repression activity in a cell and (c) positively selecting the test compound capable of mimicking the TIF1γ transcriptional repression activity in a cell.

In one particular embodiment, the method for screening a compound useful for mimicking the TIF1γ transcriptional repression activity comprising the steps of: (a) selecting a test compound by performing at least one method for identifying a compound that activates the expression of TIF1γ as described above (b) determining whether said compound mimics the TIF1γ transcriptional repression activity in a cell and (c) positively selecting the test compound capable of mimicking the TIF1γ transcriptional repression activity in a cell.

Test Compounds of the Invention:

According to one embodiment of the invention, the test compound of may be selected from the group consisting of peptides, proteins, peptidomimetics, small organic molecules, aptamers or nucleic acids. For example, the test compound according to the invention may be selected from a library of compounds previously synthesised, or a library of compounds for which the structure is determined in a database, or from a library of compounds that have been synthesised de novo.

In a particular embodiment, the test compound may be selected from small organic molecules.

As used herein, the term “small organic molecule” refers to a molecule of size comparable to those organic molecules generally sued in pharmaceuticals. The term excludes biological macromolecules (e.g.; proteins, nucleic acids, etc.); preferred small organic molecules range in size up to 2000 Da, and most preferably up to about 1000 Da.

### Transgenic Non-Human Animal Models of Inflammation

The present invention further relates to an in vivo model of inflammation: a transgenic non-human animal which is TIF1γ-deficient only in myeloid cells.

By transgenic non-human animal which is TIF1γ-deficient, it is meant a homozygous animal (i.e., the two alleles of TIF1γ are defective).

Typically suitable transgenic non-human animals are laboratory animals such as rodents, rats, mice, monkeys, dogs, rabbits, guinea pigs, goats, sheep, pigs and cattle. In one embodiment the transgenic non-human animal of the present invention is a rodent. In a more preferred embodiment the transgenic animal is a mouse.

Typically the transgenic animals according to the invention are generated using General Methods Standard molecular biology techniques known in the art (see for example U.S. Pat. No. 6,740,793, US2006/0101533).

The present invention provides a mean for identification of compounds that induce, promote or restore the resolution of inflammation, which is a defective process involved in diseases or conditions, such as inflammatory disease. Such compounds would be of significant clinical importance for treatment of inflammatory disease. The provision of the animal model according to the present invention can greatly shorten the time required for screening for such agents. Thus the present invention relates to a method for identifying a compound useful for inducing, promoting or restoring the resolution of inflammation and therefore inhibiting or treating an inflammatory disease, comprising the step of contacting a candidate compound with a transgenic animal according to the invention.

In a particular embodiment, the test compounds that have been positively selected at the end of one of the embodiments of the in vitro screening which has been described previously in the present specification may be subjected to further selection steps in view of further assaying their properties on the resolution of inflammation (e.g. protection of the mice against an LPS-induced endotoxicosis, an irradiation-induced inflammation or a DSS-induced colitis as described below).

The invention will be further illustrated by the following figures and examples. However, these examples and figures should not be interpreted in any way as limiting the scope of the present invention.

### FIGURES

FIG. 1: Quantification of local radio-induced injuries after irradiation of the snouts. A: Curve indicates the follow-up of Parkins scoring each day during twenty days after local irradiation at 16.5 Gy of C578l/6 mice. Irradiated C578l/6 mice showed an increased score 6 days after irradiation and reached a higher score 10 days after irradiation.

B: Curves indicate the following of Parkins score during the 7 days early phase of inflammation after irradiation at 16.5 Gy of Wt, Mx-Cre/Tif1γ^{-/-} or Mx-Cre/Tif1γ^{-/-} mice. Mx-Cre/Tif1γ^{-/-} mice reached a Parkins score of 4 or 5 indicating a high local inflammation 7 days after irradiation.

FIG. 2: Increased amount of circulating myeloid cells in Lyz-Cre/Tif1γ^{-/-} mice. Graphs represent the percentage of Gr1⁺CD11b⁺ cells and Ly6Ghi CD11bhi neutrophils from blood (A), bone marrow (B) and spleen (C) of Lyz-Cre/Tif1γ^{-/-} and Lyz-Cre/Tif1γ^{-/-} mice.

FIG. 3: Increased amount of circulating inflammatory monocytes in Lyz-Cre/Tif1γ^{-/-} mice. Graphs represent the percentage of Ly6Chi and Ly6Clow monocyte subsets from blood (A), bone marrow (B) and spleen (C) of Lyz-Cre/Tif1γ^{-/-} and Lyz-Cre/Tif1γ^{-/-} mice.

FIG. 4: Lyz-Cre/Tif1γ^{-/-} mice response to LPS challenge. Survival plot of Lyz-Cre/TIF1γ^{-/-} mice (n=4) and Lyz-Cre/TIF1γ^{-/-} mice (n=4) after intraperitoneal injection of LPS (0.25 mg per 20 g).

FIG. 5: Tif1γ can rescue Pai-1, flrt3, IL-33 or ifnβ genes expression in Tif1γ^{-/-} macrophages. Lin-c-kit+ progenitors from Lyz-Cre/Tif1γ^{-/-} and Lyz-Cre/Tif1γ^{-/-} mice were purified by cell sorting, cultured overnight and infected for 2 days with lentiviral vector pTRIP/ΔU3-MND-TIF1γ-1RES-GFP and control pTR1P/ΔU3-MND-GFP. Cells were then maintained in medium containing 100 ng/ml SCF and 20 ng/ml GM-CSF for a total of 7 days. For terminal macrophages differentiation, cells were then reseeded in the presence of 25 ng/ml M-CSF for 6 days. Total RNA was isolated before LPS stimulation and 4 h and 24 h after LPS stimulation. Pai-1 (A), frt3 (B), IL-33 (C) and ifnβ (D) genes expression was monitored by quantitative RT-PCR.

FIG. 6: (A) IL-33 expression is increased in TIF1γ^{-/-} macrophages activated with LPS. Quantitative RT-PCR analysis of il-33 mRNA levels in wild type and Lyz-cre/TIF1γ^{-/-} BMDM at 0, 4, 12 and 24 h following exposure to LPS (100 ng/mL). Data are the average fold changes relative to unstimulated wt BMDM (0 h)±SD. HPRT mRNA levels were used as an internal reference.

(B) LyzCre/Tif1γ^{-/-} mice response to DSS challenge. Body weight and survival plot of wild type (CTL) mice (n=8) and Lyz-Cre/TIF1γ^{-/-} mice (n=8) after treatment with DSS (2% w/v) for 6 days.

FIG. 7: High number of circulating myeloid cells in LyzCre/Tif1γ^{-/-} mice treated with DSS. Graphs represent the total number of circulating leucocytes (WBC) and the total number Gr1⁺CD11b⁺ cells and B (B220⁺) and T (CD3⁻) lymphocytes from blood of wild type (CTL) and Lyz-Cre/Tif1γ^{-/-} mice after DSS treatment for 6 days.

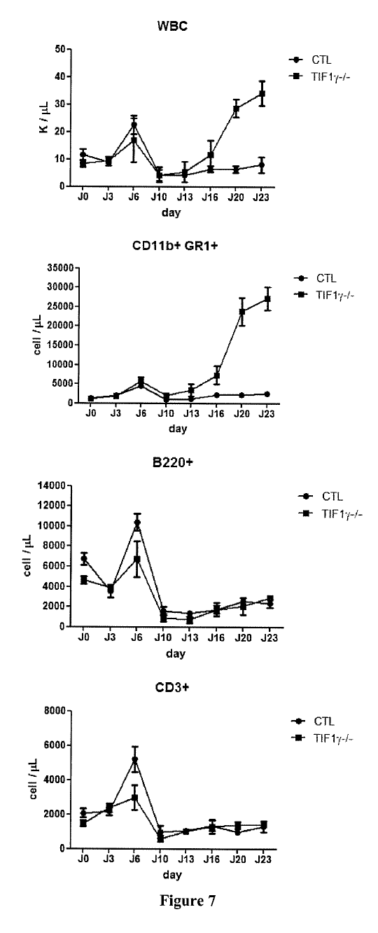

FIG. 8: High increase of circulating inflammatory cells in the LyzCre/Tif1γ^{-/-} mice 23 days after DSS challenge. Graphs represent the total number of Ly6C^{hi} monocyte subsets and of neutrophiles from blood of wild type (CTL) and Lyz-Cre/Tif1γ^{-/-} mice after DSS treatment for 6 days. Spleen of wild type (WT DSS) and LyzCre/Tif1γ^{-/-} (LyzCre/Tif1γ^{-/-} DSS) mice 23 days after DSS treatment are shown in comparison with a spleen of a non treated mouse (WT).

FIG. 9: Decreased human TIF1γ mRNA level in subset of patients with inflammatory diseases. Quantitative RT-PCR analysis of TIF1γ mRNA levels in purified circulating monocytes from 5 healthy donors (black circle), two patients with sepsis (open square) and two patients with lupus (black square), one (Lupus without inflammation) with low level of C-Reactive Protein (CRP) and one (Lupus with inflammation) with a high level of CRP. Data are the average fold changes relative to TIF1γ mRNA levels in monocytes of healthy donors. ABL mRNA levels were used as an internal reference.

### EXAMPLES

### Example 1

### TIF1γ Regulates Irradiation-Mediated Inflammation in Mice

Three wild type or Mx-Cre/TIF1γ mutant mice (Kusy et al., 2011) were housed per plastic cage and temperature (22±2° C.), lighting (12 hours) and humidity (60±10%) were under control. Animals were used in compliance with the European Union recommendations on laboratory animal care. The experimental basal diet used was liquid food (Renutryl®500, Nestlë Clinical Nutrition, France) and water. The rear paws of animals were tattooed. The oral region of mice was selectively irradiated with a single dose of 16.5 Gy, delivered by a Philips RT250 225 kV X-ray radiation source with a 0.5 mm Cu filter, at a dose rate of 0.69 Gy/min. Mice were ventrally irradiated. The whole body was lead-shielded, apart from the anterior part of the snout. After irradiation, mice were observed every day and mucosal reactions were quantified using the Parkins scoring system (Parkins et al., 1983).

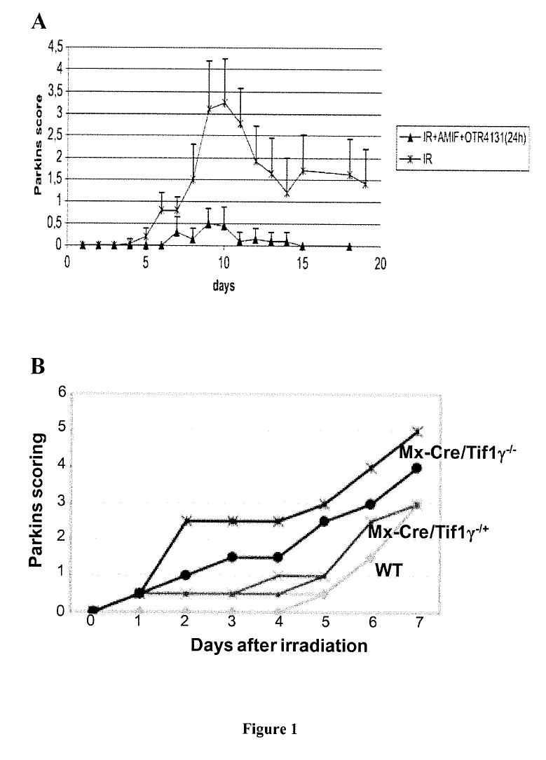

Six days after irradiation, Mx-Cre/Tif1γ^{-/-} or Mx-Cre/Tif1γ^{-/-} mice displayed numerous signs of inflammation whereas wild type mice did not show any of these signs. To quantify the degree of inflammation, the Parkins scoring of wild type, Mx-cre/Tif1γ^{-/-} and Mx-Cre/Tif1γ^{-/-} mice were determined. As shown in FIG. 1, Mx-Cre/Tif1γ^{-/-} mice reached a Parkins score of 4 or 5, seven days after irradiation indicating a major local inflammation that impaired any food uptake by the mutant mice. As a consequence, all mice were sacrificed by cervical dislocation at day 7 and histological analyses were performed on labial mucosae and lung. Tissue specimens were first washed in saline phosphate buffer (PBS) and then fixed in Finefix (Milestone, Italy) for 24 h. Then according to standard protocols, tissues were embedded in paraffin and 5 to 6 μm thick cross-sections were prepared. Tissues and inflammatory infiltration were observed on slides stained with haematoxylin and eosin (HES), while collagen and epidermis changes were measured by Masson's trichrome staining. The local inflammation was evidenced by epithelium disruption and lymphocytes and polynuclear infiltrates in the irradiated skin of the Mx-Cre/Tif1γ^{-/-} mice. We finally studied any spreading of inflammation after this local irradiation and found tissue remodelling with inflammatory cells, polynuclear, monocytes and macrophages in the lungs of the snouts irradiated Mx-Cre/Tif1γ^{-/-} mice indicating a spreading of the inflammation in the Mx-Cre/Tif1γ^{-/-} mice.

### Example 2

### A New Mouse Model to Study the Role of TIF1γ During Inflammation

The Mx-Cre/TIF1γ mutant mice have a tif1γ gene deletion in all hematopoietic cells and thus the phenotype observed previously cannot be directly related to a role of TIF1γ in the inflammatory myeloid cells. To study this role, we bred Tif1γ^{f1/f1} mice with Lyz-Cre mice (obtained from The Jackson Laboratory, Bar Harbor, Me., USA) where the Cre recombinase gene is under the regulatory sequences of the Lyz gene that is expressed only in mature myeloid cells. Lyz-Cre/Tif1γ^{f1/f1} mice were born with expected Mendelian ratios and did not display developmental abnormalities indicating that the deletion of the of γ gene in mature myeloid cells was not lethal.

Cell specific deletion of the tif1γ gene was studied in hematopoietic sub-populations by PCR analysis of genomic DNA after cell sorting. Bone marrow was extracted by flushing femurs, tibias and humerus of Lyz-Cre/Tif1γ^{-/-} and Lyz-Cre/Tif1γ^{-/-} mice with PBS. Bone marrow cell suspension was filtered through a 70 μm mesh filter to remove debris and red blood cells were lyzed with 1:10 volume of ammonium chloride solution (Stem Cell Technogies) for 10 min at room temperature. Using blue trypan solution (0.1% at volume ratio 1:1), viable cells were counted on hematocytometer. Cell Sorting experiments were performed on a BD influx sorter equipped with 5 lasers (488 nm argon, 405, 355, 561 and 635 nm) using fluorochromes. DNA was extracted using the REDExtract-N-Amp™ tissue PCR kit (Sigma-Aldrich®) from 20,000 flow sorted bone marrow cells (B-lymphocytes, neutrophiles, monocytes and Macrophage and Dendritic Precursor (MDP)) or 20,000 macrophages derived from a 7 days culture of whole bone marrow in presence of M-CSF. Genomic DNA was released from cells and, after adding neutralizing solution, 4 μl of extract are mixed with REDExtract_N_Amp PCR reaction solution (buffer, salts, dNTPs and Taq polymerase) and 3 primers to identify the wild type, floxed and excised allelesPCR were done with 35 cycles at 94° C. for 30 s, 55° C. for 30 s and 72° C. for 1 min. Deletion of the tif1γ gene was complete only in mature myeloid cells

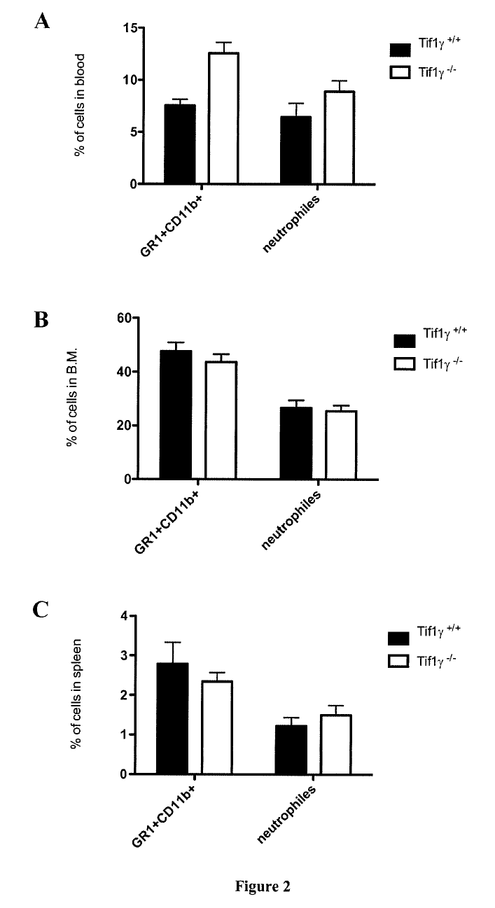

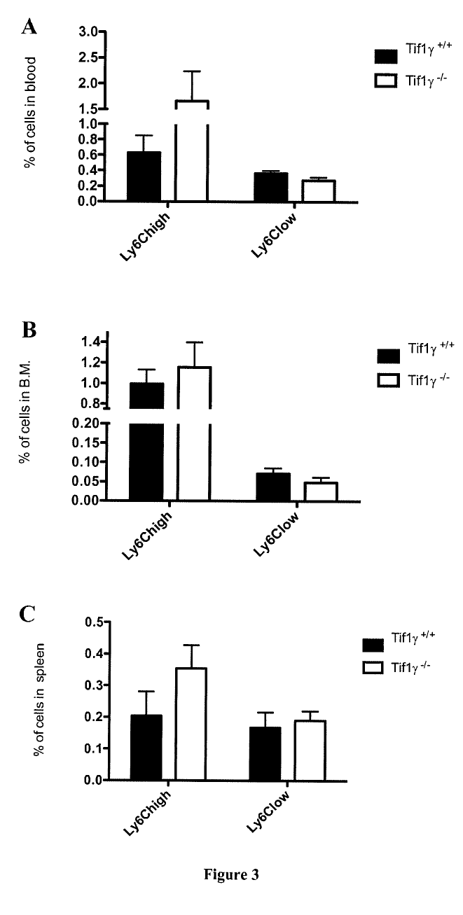

Hematopoiesis of the Lyz-Cre/Tif1γ^{-/-} mice was studied. Peripheral blood was taken by retro-orbital puncture under methoxyfluran anesthesia. Mouse bone marrow was extracted by flushing femurs, tibias and humerus of Lyz-Cre/Tif1γ^{wt/wt} and Lyz-Cre/Tif1γ^{-/-} mice with PBS. Bone marrow cell suspension was filtered through a 70 μm mesh filter to remove debris. Spleen was dissected and cell suspension was generated by gently squashing the spleen in a small volume of PBS. Spleen cells suspension was filtered using a 70 μm mesh filter in order to remove debris. Red blood cells were lyzed with a 1:10 volume of ammonium chloride solution (Stem Cell Technogies) for 10 min at room temperature. Using blue trypan solution (0.1% at volume ratio 1:1), viable cells were counted on hematocytometer. Cells (2·10⁶ to 5·10⁶) were then suspended in 100 μl of PBS and incubated with the appropriate fluorescent antibodies for 15 min at 4° C. Cell populations were phenotyped with SORPLSRII or FACScalibur (BD Bioscience). Sidescatter parameters (SSC-Area vs SSC-Width) were used for exclusion of doublets from analyses. Data were analyzed with FlowJo software (Treestar). For cell sorting, 2·10⁶ to 20·10⁶ bone marrow cells were used. Cell Sorting experiments were performed on a BD influx sorter equipped with 5 lasers (488 nm argon, 405, 355, 561 and 635 nm) using fluorochromes. The only differences found in mature myeloid cells were a two fold increased of Gr1⁺CD11b⁺ myeloid cells in the blood of TIF1γ^{-/-} mice (FIG. 2) that was correlated with a three fold increased of CD115⁺Ly6C^{hi} inflammatory monocytes in the blood of TIF1γ^{-/-} mice (FIG. 3). These results suggested a role of TIF1γ in myeloid cells during inflammation.

Lyz-Cre/TIF1γ^{-/-} Mice are Highly Sensible to LPS-Induced Endotoxicosis

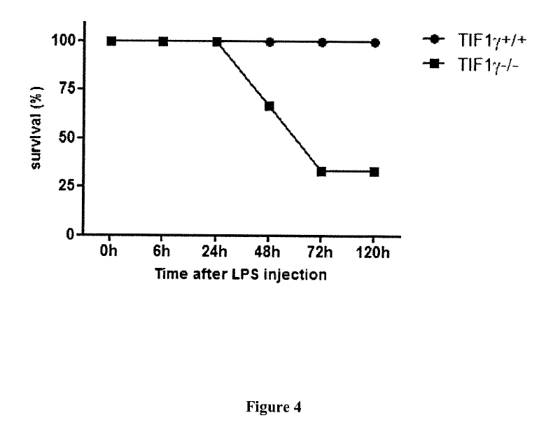

Lipopolysaccharide (LPS) injection into mice is a widely used model to study the inflammatory response associated with endotoxic shock. To investigate the role of TIF1γ during LPS-induced endotoxicosis, we administered LPS (Escherichia coli) (L3129; Sigma-Aldrich, St. Louis, Mo.) (250 mg/20 g body weight, intraperitoneal injection) and monitored routinely for signs of endotoxemia and lethality up to 120 hours after injection. All (n=4) wild type mice survived whereas only one (i.e. 25%) TIF1γ^{-/-}/LyzCre mouse survive (FIG. 4).

Mice were bled from the retro-orbital sinus at specific time points post injection, and serum was separated from the blood for cytokine measurements. A mouse inflammation cytometric bead array (CBA) kit (552364; BD Biosciences, San Jose, Calif.) was used to measure IL-6, IFN-γ, TNF-α, MCP-1 according to the manufacturer's instructions. Cytokine analyses in the scrum of LPS-treated mice revealed no significant difference in the serum level of IL-6, TNF-α, MCP-1 and IFN-γ, indicating that the sensitivity of Lyz-Cre/TIF1γ to LPS could not be accounted for by impaired pro-inflammatory responses of these cytokines post LPS injection.

### Example 3

### TIF1γ Regulates Gene Expression in Macrophages Stimulated by LPS

To study the role of TIF1γ in the transcriptional response induced by pathogen receptors, we analyzed whether lack of TIF1γ in macrophages affected the TLR-mediated regulation of proinflammatory and antimicrobial genes. Bone marrow cells from wild type and Lyz-Cre/TIF1γ^{-/-} mice (both femurs and tibias) were first suspended in 20 ml of IMDM+10% FCS+150 μM monothioglycerol and allowed to adhere for 3 hr at 37° C. in a 75 cm² flask. The non-adherent cells were then diluted to a final concentrations of 2·10⁶ cellules per ml in IMDM+10% FCS+150 μM monothioglycerol+25 ng/ml M-CSF (Miltenyi) and plated in 6 well plates (2.5 ml/well) for 7 days at 37° C., with total medium changes of the adherent cells at day 5. Bone marrow derived macrophages obtained were then treated with 100 ng/mL lipopolysaccharide (LPS) in IMDM+2.5% FCS and total RNA was isolated 0, 4 h, 12 h and 24 h post-LPS treatment with RNeasy micro kit (Qiagen). RNA quality was assessed on Agilent 2100 Bioanalyzer and RNA were used for microarrays analysis (Mouse GE 8×60K, Agilent) at IGFR platform (France). Before LPS treatment, less than 100 genes displayed a different expression in wild type and Lyz-Cre/TIF1γ^{-/-} macrophages. When these macrophages were stimulated with LPS, both genes with fast induction kinetics and genes with a slower more gradual induction were differentially regulated in wild type and Lyz-Cre/TIF1γ^{-/-} mouse bone marrow-derived macrophages (Table 1). Finally, twenty-four hours after LPS treatment, numerous genes that are repressed during the resolution of inflammation in macrophages were not repressed in Lyz-Cre/TIF1γ^{-/-} macrophages indicating a role of TIF1γ in the resolution of inflammation (Table 1).

TABLE 1
Summary of data obtained from microarray analyses of Lyz-Cre/TIF1γ^{-/-}
macrophages without LPS treatment (0 h) and 4, 12 and 24 hours after LPS treatment.

| | | | |
|---|---|---|---|
| 0 h | 4 h | 12 h | 24 h |

Up-regulated genes

| | | | | |
|---|---|---|---|---|
| Transcription factors | Neo1 | ASB4, Btbd11, EPAS1, | Foxo3, Hexim2, Neo1 | Foxo3, Neo1, Nfatc4, |
| | | GMNN, Neo1, Nostrin | | Pou3F1 |
| Inflammatory | Stfa2, Stfa3, Stfa1, stfa211 | IL5Ra, Klkb1, Lair1, Lat2, | P2rx7, P2ry12, SPP1, | CD300lf, Ctsk, Gsto1, |
| regulators | | P2rx7, P2ry12, TNFSF4 | TNFSF4 | P2rx7, P2ry12, P2ry13, |
| | | | | Ptgis, Spsb4, TNFSF4 |
| Cytokines and | | CXCL14, IFNb, IL33 | CCL17, CCL7, CXCL14, | CCL6, CXCL14, IL33, |
| chemokines | | | IL33, IFNb | IFNb |
| Migration/adhesion | CADM1, Flrt3 | CCND1, CTGF, Flrt3, | AGT, ANGPT1, | AGT, ANGPTL3, |
| | | Esr1, Lipg, LAMC2, | ANGPTL3, CD36, CHRD, | ARHGAP8, CD36, |
| | | MCM7, MMP10, PGF, | COL18A1, CTTN, Esr1, | Cadm1, CamK2a, Flrt3, |
| | | qPCT, TNN, TPM1 | Flrt3, GSN, ICAM2, | Esr1, Lipg, MMP12, |
| | | | MMP12, NOX4, qPCT, | qPCT, RGS1, PAI1, |
| | | | RGS1, Serpine1, | S100A4, SPP1, TGM2, |
| | | | S100A4, TGM2, VEGFA | VEGFA |
| Proliferation | | AMACR, BIRC5, | CD180, CDKN1A, | |
| | | BLOC1S2, BUB1, | KCNN4 | |
| | | CDC25C, CDK14, | | |
| | | CKS1B, COMMD5, | | |
| | | CTGF, DTYMK, FOLR1, | | |
| | | GLMN, GPN3, IL5RA, | | |
| | | MKI67, NDUFAF4, PGF, | | |
| | | PTGER2, RAD51, | | |
| | | TDGF1, WRD12 | | |

Down-regulated genes

| | | | | |
|---|---|---|---|---|
| Transcription factors | | BHLHE40, HHEX, MAF, | BHLHE40, EGR1, HHEX, | BHLHE40, EGR1, |
| | | MNT, NAB2 | HOXD13, ID3, SATB1, | HOXD1, KLF3, ID3, |
| | | | SMAD6, SPIC, STAT4 | SATB1, SMAD6 |
| Inflammatory | PLA2G7, Saa3 | AOC3, CSF2Rb, GZMK, | FOS, GZMK, THBS1, | GZMK, IFITM1, IL18R1, |
| regulators | | IL12RB1, Nos2, SELP, | Serpina1, THY1 | Pdc5a, Serpina1, THBS1, |
| | | THY1 | | THY1 |
| Cytokines and | | CCL8, CSF3, CXCL9, | CCL25, CSF2, CSF3 | CCL25, CSF2, CSF3, |
| chemokines | | IFNg | | CX3CR1, CXCL5, |
| | | | | CXCR2, IFNg |
| Migration/adhesion | VCAM1 | BIRC3, CDKN1a, CNR2, | GAB1, MET, | CD74, CDKN1a, ENPP2, |
| | | DLG1, ELN, FN1, FBN1, | MMP2, NCAM1, NR4A1, | EPHBP2, GAB1, GJA1, |
| | | ITGA5, ITGAL, MMP14, | NRG1, PARP9, | ITGA9, ITGAL3, ITGB3, |
| | | PROCR, PRRCE, | P2ry2, PRKCB, RAP2A | MET, MMP2, NCAM1, |
| | | S100A2, SELP | | NR4A1, NRG1, OSM, |
| | | | | P2ry2, PARP9, PLA2G7, |
| | | | | PPBP, PRKCB, RAP2A, |
| | | | | ZAP70 |
| Proliferation | | BCL2, BDNF, KCNA3, | BTG3, HK2, IGF2BP1, | |
| | | Lin28A, MYLK, | IGF2BP3, OVOL2, | |
| | | RALGDS | RARG, RBP3 | |

To characterize the role of TIF1γ during the LPS activation of macrophages, the pai-1 (serpin1) and ifnβ genes expression and chromatin structure were studied during the LPS-mediated activation of macrophages. PAI-1 (type 1 Plasminogen Activator Inhibitor) is a key regulator for fibrinolysis and coagulation by counteracting plasminogen activators and act as an acute-phase protein during inflammation and sepsis. The pai-1 gene is activated early after LPS treatment of macrophages and repressed during the resolution of inflammation. Ifnβ is a well known player of inflammation. Its gene is activated early after LPS treatment of macrophages and repress during the resolution of inflammation. In wild type and Lyz-Cre/TIF1γ^{-/-} macrophages, the pai-1 and the ifnβ genes were activated after LPS treatment but, in Lyz-Cre/TIF1γ^{-/-} macrophages, the pai-1 and the ifnβ genes were not repressed at the end of macrophages activation. TIF1γ and PU.1 binding together with the chromatin structure of the pai-1 and the ifnβ genes were studied by chromatin immunoprecipitation. Bone marrow derived macrophages untreated or treated with LPS were fixed with 1% formaldehyde for 10 min at 37° C. and lysed in SDS lysis buffer (50 mM Tris pH8, 10 mM EDTA, 1% SDS, protease inhibitor cocktail) for 10 min at 4° C. The chromatin was sheared by sonication to obtain DNA fragments with an average length of 200-800 bp using a Bioruptor (Diagenode, cycle conditions: 15 sec ON/30 sec OFF, 15 min). Lysates were diluted 10 times in IP dilution buffer (16.7 mM Tris pH8, 167 mM NaCl, 1.2 mM EDTA, 1.1% Triton X100, 0.01% SDS, protease inhibitor cocktail) and immunoprecipitations were performed at 4° C. overnight. 1 μg of antibodies against PU.1 (Santa Cruz), RNA poIII (Santa Cruz) and H3K4me3 (Millipore) was used to precipitate an equivalent of 0.5×10⁶ cells; 1 On of antibody against TIF1γ (Bethyl) were used to precipitate an equivalent of 8×10⁶ cells. A negative control IgG antibody was included in the ChIP assay. Immunoprecipitated chromatin was collected using protein A agarose/Salmon Sperm DNA beads, washed and eluted with 1% SDS, 50 mM Tris pH8, 10 mM EDTA. Crosslinking was reversed with 0.2M NaCl at 65° C. overnight. The chromatin was then incubated with 20 μg of Proteinase K (Invitrogen) for 1 h at 45° C. and DNA was purified by phenol/chloroform extraction and ethanol precipitation. Quantitative PCR reactions were performed using SYBR Green PCR Master Mix (Applied Biosystems) and specific primers amplifying a regulatory region (−4 kb relative to the transcriptional start site) and the promoter of the pai-1 gene. Data were normalized to input signal and reported to IgG values. The results were represented as fold enrichment over the signal obtained in a control sequence in the β-globin gene. TIF1γ was bound together with PU.1 to the −4 kb pai-1 gene enhancer and without PU.1 to the pai-1 promoter in non-activated and LPS-activated macrophages. Twenty-four hours after LPS treatment a 3 to 4 fold increased binding of TIF1γ was found but only on the promoter of the pai-1 gene. In Lyz-Cre/TIF1γ^{-/-} macrophages, no difference in the RNA poIII occupancy and chromatin structure of the pai-1 gene was found before LPS activation and 4 hours after activation but 12 and 24 hours after LPS treatment, RNA poIII occupancy and methylation of lysine 4 of histone 3 (H3K4me3) was 4 to 8 fold higher in TIF1γ^{-/-} macrophages. As for the ifnβ gene, TIF1γ was bound together with PU.1 to the −15 kb pal-1 gene enhancer and to the ifnβ promoter in non-activated and LPS-activated macrophages. Twenty-four hours after LPS treatment a 3 to 4 fold increased binding of TIF1γ was found on the promoter of the ifnβ gene. In Lyz-Cre/TIFγ^{-/-} macrophages, no difference in the RNA poIII occupancy and chromatin structure of the ifnβ gene was found before LPS activation and 4 hours after activation but 12 and 24 hours after LPS treatment, RNA poIII occupancy and methylation of lysine 4 of histone 3 (H3K4me3) was 10 fold higher in TIF1γ^{-/-} macrophages.

### Example 4

### Ectopic TIF1γ Expression can Rescue Gene Expression in LPS-Activated TIFγ^{-/-} Macrophages

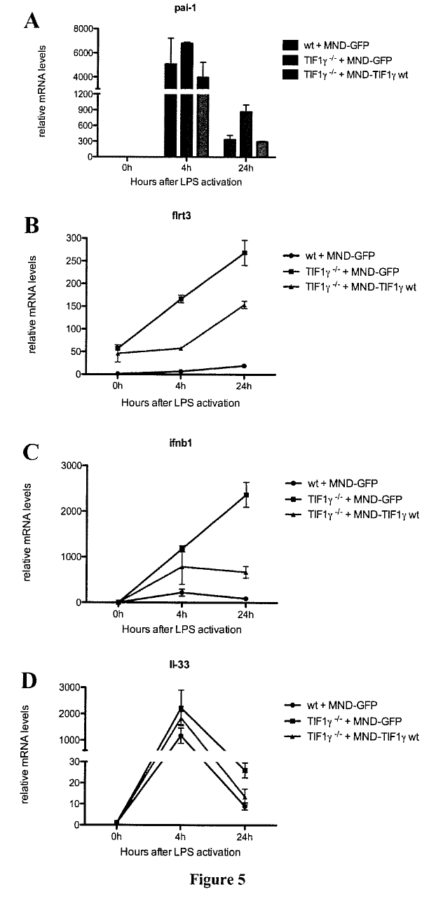

The role of TIF1γ in the expression of genes transcriptionally regulated during LPS-mediated activation of macrophages was studied after infection of Lin⁻c-kit⁻ progenitors from wild type or Lyz-Cre/TIF11γ^{-/-} bone marrow with lentivirus that contained tif1γ cDNA under the control of the MND promoter. Lin⁻c-kit⁺ progenitors were purified by cell sorting, cultured overnight and infected for 2 days in IMDM medium supplemented with 15% BIT 9500 (StemCells Technologies), in the presence of 20 ng/mL IL-3, 100 ng/mL SCF, 10 ng/mL 11-6, 100 ng/mL Flt-3-ligand, 100 ng/mL TPO. Cells were transduced with lentiviral vector pTRIP/ΔU3-MND-TIF1γ-IRES-GFP and control pTRIP/ΔU3-MND-GFP in the presence of 5 μg/mlprotamine sulfate at final MOI of 10. For myeloid expansion, cells were then maintained in medium containing 100 ng/ml SCF and 20 ng/ml GM-CSF for a total of 7 days. For terminal macrophages differentiation, cells were then reseeded in the presence of 25 ng/ml M-CSF for 6 days. Total RNA was isolated before LPS stimulation and 4 h and 24 h after LPS stimulation. TIF1γ could rescue the regulated expression of some but not all genes deregulated in Lyz-Cre/TIF1γ^{-/-} macrophages indicating the pai-I, flrt3, IL-33 or ifnβ genes as directs targets of TIF1γ (FIG. 5).

### Example 5

Lyz-Cre/TIF1γ^{-/-} Mice are Sensible to DSS-Induced Colitis.

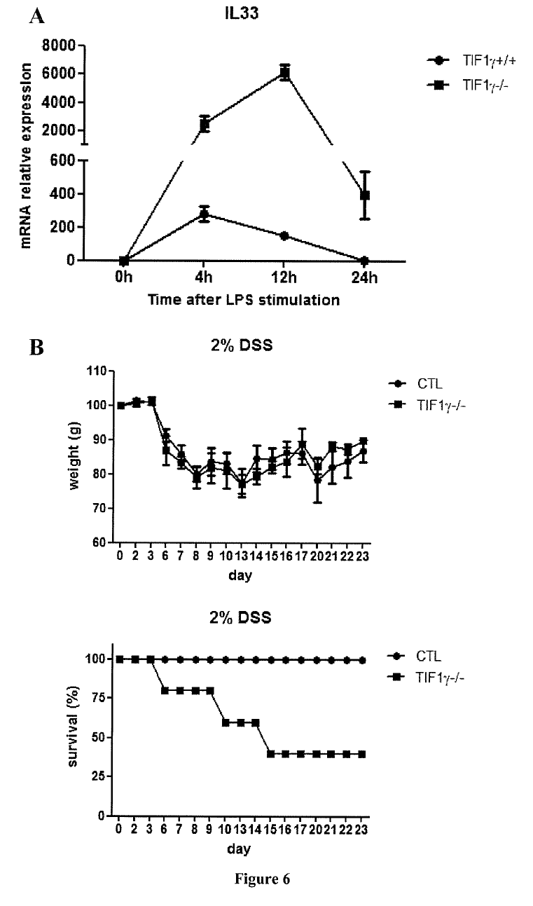

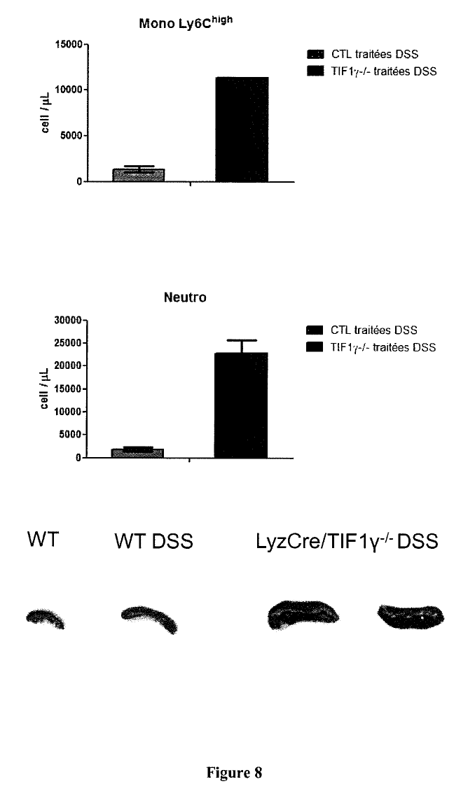

The intestinal injury caused by dextran sodium sulfate (DSS) in mice shares some characteristics with ulcerative colitis (UC) in human patients as both are characterized by diffuse mucosal inflammation, superficial ulceration and goblet cell depletion. In human UC patients, IL-33 expression is highly up-regulated and IL-33-deficient mice are protected from DSS-induced intestinal immunopathology (Oboki et al., 2010). As IL-33 was a direct TIF1γ target gene and as iI-33 mRNA level was increased in Lyz-Cre/TIF1γ^{-/-} macrophages stimulated with LPS (FIG. 6A), we studied DSS-induced colitis in Lyz-Cre/TIF1γ^{-/-} mice. DSS (41 kDa) (ICN Biomedical) was used to supplement the drinking water of wild type and Lyz-Cre/TIF1γ^{-/-} mice for 6 days as 2% (w/v) solution (Wirtz et al., 2007). Fresh solution was replaced at day 3. After day 6, mice returned to normal water and monitored for an additional 17 days. Body weight and survival curved showed that Lyz-Cre/TIF1γ^{-/-} mice were highly sensitive to this DSS-induced colitis and that this sensitivity was not accounted for by body weight loss (FIG. 6B). We monitored the circulating leucocytes and found a high increased of myeloid cells in the surviving Lyz-Cre/TIF1γ^{-/-} mice whereas no difference in circulating B or T lymphocytes could be detected between wild type and Lyz-Cre/TIF1γ^{-/-} mice (FIG. 8). This increased number of myeloid cells was accounted for by a high increase of circulating inflammatory cells such as monocytes Ly6C^{high} and neutrophiles and was associated with a splenomegaly at day 23 (FIG. 8). These results indicate that the inflammatory response initiated with DSS treatment was not resolved in Lyz-Cre/TIF1γ^{-/-} mice.

### Example 6

### Mutation in TIF1γ Gene Leading to a Cytoplasmic Localization of TIF1γ

Wild type TIF1γ or a mutant of TIF1γ that did not contain the COOH-bromodomain harboring the nuclear localization sequence of TIF1γ was introduced in NIH3T3 cells and their sub-cellular localization was determined by immunofluorescence. Deletion of the COOH-bromodomain results in a cytoplasmic localization of TIF1γ indicating that IHC might be used to detect mutations associated with the expression of a truncated TIF1γ protein if the truncation deletes the COOH-bromodomain.

### Example 7

### TIF1γ, a New Biomarker of Acute Inflammation

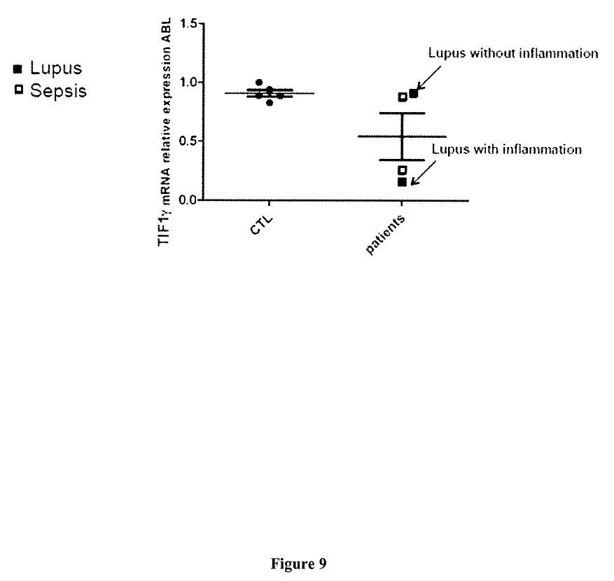

Peripheral monocytes from two patients with sepsis, one patient with inflammatory lupus (high level of C-Reactive protein (CRP)) and one patient with non inflammatory lupus (low level of CRP) were purified using anti-CD14 beads and magnetic columns and total RNA was extracted from these monocytes. As control, monocytes from five healthy donors were purified and total RNA was extracted. Quantitative RT-PCR was performed to determine the mRNA level of TIF1γ. As shown in FIG. 9, the mRNA level of TIF1γ was decreased in half of the patients with acute inflammation suggesting that TIF1γ might be used as a biomarker of acute inflammation.

### REFERENCES

Throughout this application, various references describe the state of the art to which this invention pertains. The disclosures of these references are hereby incorporated by reference into the present disclosure.
Aucagne R, Droin N, Paggetti J, Lagrange B, Largeot A, Hammann A, Bataille A, Martin L, Yan K P, Fenaux P, Losson R, Solary E, Bastie J N, Delva L. Transcription intermediary factor 1γ is a tumor suppressor in mouse and human chronic myelomonocytic leukemia. J Clin Invest. 2011 June; 121(6):2361-70.
Kusy S, Gault N, Ferri F, Lewandowski D, Barroca V, Jaracz-Ros A, Losson R and Romeo P H (2011) Adult hematopoiesis is regulated by TIF1γ, a repressor of TALI and PU.1 transcriptional activity. Cell Stem Cell, 4: 412-425
Oboki K, Ohno T, Kajiwara N, Arae K, Morita H, Ishii A, Nambu A, Abe T, Kiyonari H, Matsumoto K, Sudo K, Okumura K, Saito H, Nakae S. IL-33 is a crucial amplifier of innate rather than acquired immunity. Proc Natl Acad Sci USA. 2010 Oct. 26; 107(43):18581 Parkins C S, Fowler J F, Yu S (1983) A murine model of lip epidermal/mucosal reactions to X-irradiation. Radiother and Oncol 1: 159-165
Wirtz S, Neufert C, Weigmann B, Neurath M F. Chemically induced mouse models of intestinal inflammation. Nat Protoc. 2007; 2(3):541-6.

1. (canceled)

2. A method of preventing or treating an inflammatory disease in a subject in need thereof, comprising administering to the subject a therapeutically effective amount of a TIF1γ-encoding polynucleotide or a TIF1γ polypeptide.

3. The method according to claim 2, wherein the inflammatory disease is selected from the group consisting of rheumatoid arthritis (RA), inflammatory bowel disease (IBD), diabetes, sepsis, allergic inflammation, asthma, chronic obstructive pulmonary disease (COPD), atherosclerosis, multiple sclerosis and systemic lupus erythematosus (SLE).

4. A method for screening a compound capable of promoting and/or restoring the resolution of inflammation, comprising the step of identifying a compound that activates the expression of TIF1γ or identifying a compound that mimics TIF1γ transcriptional activity.

5. A transgenic non-human animal which is TIF1γ-deficient in myeloid cells.

6. A method for diagnosing or predicting an inflammatory disease, or a risk of an inflammatory disease, in a subject, said method comprising detecting a mutation in the TIF1γ gene in a sample obtained from said subject, wherein the presence of a TIF1γ mutation is indicative of an inflammatory disease or of a risk of an inflammatory disease.

7. A method for diagnosing or predicting an inflammatory disease, or a risk of an inflammatory disease, in a subject, said method comprising (i) detecting the level of expression of the TIF1γ gene in a sample obtained from said subject, and (ii) comparing said level of expression with a predetermined reference value, wherein a decrease in the level of expression of the TIF1γ gene is indicative of an inflammatory disease or of a risk of an inflammatory disease.

8. The method according to claim 6, wherein the inflammatory disease is selected from the group consisting of RA, IBD, diabetes, sepsis, allergic inflammation, asthma, COPD, atherosclerosis, multiple sclerosis and SLE.

9. (canceled)

10. The method according to claim 7, wherein the inflammatory disease is selected from the group consisting of RA, IBD, diabetes, sepsis, allergic inflammation, asthma, COPD, atherosclerosis, multiple sclerosis and SLE.
